(19) United States
(12) Patent Application Publication
(10) Pub. No.: US 2006/0188390 A1
(43) Pub. Date: Aug. 24, 2006

(54) Fumigation tarp

(76) Inventors: Richard Merle Galloway, Midlothian, VA (US); David Charles Jones, Midlothian, VA (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/360,719

(22) Filed: Feb. 23, 2006

Related U.S. Application Data
(60) Provisional application No. 60/655,940, filed on Feb. 24, 2005.

(51) Int. Cl. A61L 2/20

(52) U.S. Cl. 422/32

(57) ABSTRACT

A composite sheet material comprising a substantially gas-impermeable laminate made of a reinforcement scrim and a flexible sheet material that is extrusion-coated with a thermoplastic polymer resin particularly useful as a fumigation tarp having extremely low chemical gas permeability, high strength to weight ratio and good durability.

### BACKGROUND OF THE INVENTION

1. Field of the Invention

This invention is directed to a flexible substrate for use as a fumigation tarp for use in fumigation processes for the purposes of sealing fumigation gases within an enclosed space.

2. Description of the Related Art

Fumigation tarpaulins (tarps) are known for use in processes in which a certain volume is filled with fumigant gases that must be contained for a minimum period of time in order for the gases to exterminate the past therein. Such processes include the fumigation of the interior of buildings, the fumigation of fruits and vegetables within containers or transportation vehicles, and the fumigation of soil such as on a golf course.

During processes to fumigate the interior of buildings, to exterminate insects, etc., tarps are laid over the entire building, generally installing beginning from the roof. Individual tarps are joined by rolling two tarp edges together to make a rolled seam, which is clipped together to prevent unraveling.

Among the gases that are used to fumigate buildings are methyl bromide (CH₃Br), sulfuryl fluoride (F₂O₂S), and phosphine (PH₃).

U.S. Pat. No. 4,033,367 discloses a tent fumigation apparatus for placement over a building to allow fumigation of the building by introducing fumigating gas into the tent. The tent comprises a gas tight canvas, which is placed over a building to be fumigated. Sections of the tent are folded and tied off to adjust the shape of the tent to the building. The bottom of the tent is made relatively gas tight by the use of the tube structure that may be a water-filled hose or sand-filled bags. The top of the tent is sealed and the seams are of a relatively gas-tight construction. In operation, a vacuum pump is used to reduce the pressure inside of the tent, thus collapsing it around the building. Then fumigating gas is introduced into the tent and the building and remains until fumigation is complete.

PCT Patent Publication No. WO 2002098219 A1 discloses a method and apparatus for fumigating perishable goods, such as fruit, using a canopy and floor seal. The canopy is preferably a tarp or cover comprised of a substantially gas impermeable material, such as polyethylene, preferably comprised of a material having a melting point safely below the trigger temperature for a typical warehouse sprinkler system.

It would be desirable to have a strong, thin, lightweight fumigation tarp having extremely low gas permeability and good abrasion resistance and durability.

### DETAILED DESCRIPTION OF THE INVENTION

The building fumigation tarp of the present invention comprises a multilayer flexible composite sheet material having good strength, lightweight and very low air permeability. The composite sheet comprises a first layer of a strong, durable, lightweight sheet material. Examples of materials useful as the first layer of the composite sheet include nonwovens, such as spunbond polyesters, such as that sold under the tradename Reemay®) and spunbond polyolefins such as spunbond polypropylene (such as that sold under the trade name Typar®) and spunbond polyolefin film-fibrils of the type disclosed in U.S. Pat. Nos. 3,169,899 and 3,532,589, the contents of which are both incorporated by reference herein. A commercial nonwoven polyethylene film-fibril sheet product that is particularly suitable as the composite sheet of the invention is Tyvek® flash-spun polyethylene plexifilamentary film-fibril sheet, available from E. I. du Pont de Nemours and Company, Wilmington, Del. (DuPont). Tyvek® sheet is flexible, lightweight, and strong. Other nonwoven materials can be used as the first layer in the composite sheet of the tarp, including SMS (spunbond-meltblown-spunbond) materials and composite nonwoven materials including bicomponent fibers, microfibers, and nanofibers.

One sheet product for use in the invention is TYVEK® 1461-L sheet, due to its advantageous Elmendorf tear strength of between 14.1 and 15.7 Newtons and a tensile strength of about 25 N/cm. TYVEK® 1461-L sheet has a thickness of between 0.1 and 0.25 mm and a basis weight of about 59 g/m² (1.74 oz/yd²). TYVEK® 1461-L sheet is made of high-density polyethylene, which makes it readily recyclable.

In order to improve the resistance of TYVEK® sheet material to UV degradation, a UV stabilizer may be added to the polyethylene from which TYVEK® 1461-L sheet is flash-spun. One UV stabilizer that has been employed to stabilize TYVEK® sheets against UV degradation is the hindered amine stabilizer Tinuvin 622 made by Ciba-Geigy of Hawthorn, N.Y. Antioxidants (such as Irganox 1010 also made by Ciba-Geigy) and acid neutralizers (such as calcium stearate) can also be added to the sheet in order to reduce degradation during extended weathering. Hydroxylamine stabilizers may also employed to stabilize TYVEK® sheets against UV degradation.

The second layer of the composite sheet is a thermoplastic, flexible open mesh reinforcement scrim. The reinforcement scrim material may be a cross-laminated open mesh fabric that is at least 25% open, and has a thickness in the range of 0.07 mm to 0.25 mm. A suitable reinforcement scrim material has a basis weight in the range of 15 g/m² (0.44 oz/yd²) to 60 g/m² (1.77 oz/yd²), an Elmendorf tear strength of at least 2.5 Newtons, a tensile strength of at least 20 N/cm (11.4 lbs/in) and an elongation of less than about 30%. A scrim material that has been found especially suitable for use in the composite sheet of the invention is a cross-laminated reinforcing scrim material that is sold under the name CLAF® by Amoco Nisseki CLAF, Inc. of Atlanta, Ga., and is more fully described in U.S. Pat. No. 5,182,162, which is hereby incorporated by reference. CLAF® is a registered trademark of Nippon Petrochemicals Company, Ltd. CLAF® is made from polyethylene film that has been fibrillated with the resulting fibers spread in two transverse directions at a strand count of 2 to 4 strands per centimeter. The spread fibers are cross-laminated by heat to produce a nonwoven open mesh fabric with a thickness of 75 to 125 micrometers and with substantially equivalent strength in the machine and cross directions. The polyethylene fibers of the reinforcing material are preferably include low-density polyethylene that has a lower melting temperature than the polyethylene of the flash-spun TYVEK®.

The first and second layers are laminated together by any known laminating means, such as extrusion lamination or thermal lamination. In one embodiment of the invention, the layers are extrusion laminated by extruding a layer of thermoplastic polymer on one surface of the first layer having a thickness of between 0.5 and 1.5 mil and applying the second layer to the extruded layer and passing the layers through a nip. Optionally, a second extrusion layer can be extruded on the outer surface (the surface opposite the extruded surface) of the first layer. Having two layers of extruded polymer provides greater durability, abrasion resistance and longer tarp life, as well as lower MVTR and permeability.

In another embodiment of the invention, the first and second layers are placed adjacent each other, and passed through an extrusion lamination process in which a layer of polymer is extruded on the outer surface of the first layer. Optionally, the first and second layers are first thermally laminated together, and then a layer of thermoplastic polymer is extruded on the outer surface of the first layer.

The thermoplastic polymer forming the extruded layer may be polyethylene or some other extrusion resin. An ethylene methacrlyic acid copolymer resin (sold under the trade name Surlyn®, available from DuPont) provides the composite sheet with good abrasion resistance.

The chemical gas barrier of the tarp of the invention is extremely low. Gas barrier is measured by ASTM F739 (gas phase) at room temperature and is reported in gms/m²/hour.

The layer(s) of extruded polymer and first and second layers are preferably the lightest weight possible, while still providing sufficiently low air permeability to perform the function of sealing the fumigation gases within the tarp when in use.

The combination of light weight, relatively low thickness, strength, and chemical barrier of the tarp of the invention provide important advantages in the applications in which the tarp is used. For one, the light weight of the tarp enables the tarp to be handled more safely than heavier incumbent tarps. This is especially important for workers installing the tarps on building roofs. For another advantage, the low thickness of the tarp of the invention enables multiple tarps to be rolled together to form rolled seams which are tighter than the rolled seams formed with the current incumbent tarps, and thus less likely to leak fumigant gases. Another advantage that is believed to be provided by the tarp of the invention is that sewn seams are less likely to leak fumigant gases because the fibers of the nonwoven layer tend to self-seal around the holes formed by the sewing needles. For these reasons, the tarps of the invention are less likely to leak gas and less fumigant gases need to be used to attain the same level of effectiveness.

### TEST METHODS

In the description above and in the non-limiting examples that follow, the following test methods were employed to determine various reported characteristics and properties. ASTM refers to the American Society for Testing and Materials, and AATTC refers to the American Association of Textile Chemists and Colorists.

Basis Weight was determined by ASTM D-3776, which is hereby incorporated by reference, and is reported in g/m². The basis weights reported for the examples below are each based on an average of at least twelve measurements made on the sample.

Tensile Strength and Work to Break were determined by ASTM D-1682, Section 19, which is hereby incorporated by reference, with the following modifications. In the test, a 2.54 cm by 20.32 cm (1 inch by 8 inch) sample was clamped at its opposite ends. The clamps were attached 12.7 cm (5 in) from each other on the sample. The sample was pulled steadily at a speed of 5.08 cm/min (2 in/min) until the sample broke. The force at break was recorded Newtons/cm as the breaking tensile strength. The area under the stress-strain curve was the work to break.

Grab Tensile Strength was determined by ASTM D 1682, Section 16, which is hereby incorporated by reference, and is reported in Newtons.

Hydrostatic Head measures the resistance of a sheet to the penetration by liquid water under a static load. A 316 cm² sample is mounted in an SDL Shirley Hydrostatic Head Tester (manufactured by Shirley Developments Limited, Stockport, England). Water is pumped against one side of a 102.6 cm² section of the sample until the sample is penetrated by water. The measured hydrostatic pressure is reported in centimeters of water. The test generally follows AATTC 127-1985, which is hereby incorporated by reference. The hydrostatic head values reported for the examples below are each based on an average of at least six measurements made on the sheet.

Moisture Vapor Transmission Rate (MVTR) is determined by ASTM E96, Method B, which is hereby incorporated by reference, and is reported in g/m²/124 hr.

### EXAMPLE

A 33 g/m² basis weight cross laminated polyethylene open mesh nonwoven reinforcement scrim (commercially available as CLAF® from Atlanta Nisseki CLAF, Inc., Kennesaw, Ga.) was laminated to a 58 g/m² basis weight polyethylene plexifilamentary film-fibril sheet of TYVEK®. These two layers were joined by a heat lamination process as described in U.S. Pat. No. 5,763,336 to Jones et al. An extruded layer of low density polyethylene with a basis weight of 18 g/m² commercially available from Westlake Petrochemical Corporation, Sulphur, La. was placed on top of the reinforcement scrim. The composite sheet had a total basis weight of about 109 g/m². The composite sheet had a final thickness of 250 micrometers, a machine direction tensile of 69 N/cm, a machine direction elongation of 15%, a machine direction work-to-break of 3.9 Nm, a machine direction trap tear of 136 N, a machine direction Elmendorf tear of 12 N, and a Spencer puncture of 213 N.

A large square of the composite sheet was sewn into a tarp using a dual stitch seam. A chain stitch was used, as commonly used in such applications. A chain stitch resembles a chain link and is formed with one thread fed from the bottom side of the fabric and is done on a manual or computerized machine with a hook that functions like a needle.

It was found that the inventive tarp lost less gas than a conventional 1.5 mm thick, 610 g/m² vinyl tarp reinforced with an open scrim. It is believed that because the tarp of the invention is thinner than the vinyl incumbent, this results in less bulky seams, and therefore less gas leakage.

The building fumigation tarp of the invention is a laminated composite sheet that is lightweight, strong, durable, and impermeable. The composite sheet material of the invention is especially useful in end use applications such as sealing buildings for building fumigation. It will be apparent to those skilled in the art that modifications and variations can be made in the building fumigation tarp of this invention. The invention in its broader aspects is, therefore, not limited to the specific details or the illustrative examples described above. Thus, it is intended that all matter contained in the foregoing description, drawings, and examples shall be interpreted as illustrative and not in a limiting sense.

What is claimed is:

1. A fumigation tarp comprising a composite sheet material comprising:
a layer of a flexible sheet material having a tensile strength of at least 20 N/cm, a work to break of at least 0.6 Nm, and a grab tensile of at least 150 N,
a layer of a thermoplastic open mesh reinforcement scrim laminated to the first layer; and
a layer of polymer extruded on the scrim layer with the extruded layer having a thickness no less than 12 micrometers,
wherein the layers are laminated to form a composite sheet having a thickness of no greater than 380 micrometers, a tensile strength of at least 65 N, an Elmendorf tear resistance of at least 10 N and a basis weight of no greater than 135 g/m².

2. The fumigation tarp of claim 1, wherein the composite sheet has a gas permeation rate of no more than 1.0 gram/m²/hour.

3. The fumigation tarp of claim 1, wherein the flexible sheet is a polyethylene plexifilamentary film-fibril strand material.

4. The fumigation tarp of claim 1, wherein the polymer extruded on the flexible sheet is low-density polyethylene.

5. The fumigation tarp of claim 1, wherein a layer of polymer is extruded onto the flexible sheet layer with the extruded layer having a thickness of no less than 12 micrometers.

6. A method for fumigating a building using the fumigation tarp of claim 1 or claim 5.
